(19) United States
(12) Patent Application Publication
(10) Pub. No.: US 2006/0147339 A1
(43) Pub. Date: Jul. 6, 2006

(54) Methods and apparatus for ultraviolet sterilization

(76) Inventors: Charles Eric Hunter, Jefferson, NC (US); Drew G. Narayan, Durham, NC (US); Laurie E. McNeil, Chapel Hill, NC (US); John H. Hebrank, Durham, NC (US)

Correspondence Address: Vollrath & Associates, #531, 588 Sutter Street, San Francisco, CA, 94102, US

(21) Appl. No.: 11/286,856

(22) Filed: Nov. 26, 2005

Related U.S. Application Data
(63) Continuation of application No. 11/097,111, filed on Apr. 1, 2005.
Application No. 11/097,111 is a continuation of application No. PCT/US03/30553, filed on Sep. 30, 2003.
(60) Provisional application No. 60/414,626, filed on Oct. 1, 2002.

(51) Int. Cl. A61L 2/10

(52) U.S. Cl. 422/24; 250/455.11

(57) ABSTRACT

Methods and apparatus for ultraviolet sterilization are provided. The apparatus can include a housing having an opening through which objects can be inserted for sterilization, a killing chamber having an inner surface that is substantially reflective at ultraviolet wavelengths; and at least one ultraviolet radiation source inside the housing. The methods and apparatus are well suited for medical tools, dental tools, body art tools, and baby feeding related objects.

### FIELD OF THE INVENTION

This invention is directed to methods and apparatus for rapidly sterilizing objects, and more particularly to methods and apparatus for sterilizing medical tools, dental tools, body art tools, and breast and bottle-feeding equipment using ultraviolet radiation.

### BACKGROUND OF THE INVENTION

Most common bacterial and cellular organisms that can cause sickness and disease in humans can be killed with moderate doses of ultraviolet light having a wavelength between about 260 nm and about 280 nm.

Germicidal infection and patient-to-patient contamination remains a major problem in hospitals and other hygienic environments. For example, ear thermometers, stethoscopes, and other devices that are reused, or not kept in sterile conditions, can spread germs between hospital patients. Also, according to one estimate, infection occurs in 3% of all surgical procedures. Some of these infections are believed to occur from non-sterile surgical tools. Moreover, the problem appears to be worsening as organisms become resistant to antibiotics.

One conventional sterilization method involves heating tools to high temperatures using dry heat, boiling water, and steam baths. These methods, however, consume a substantial amount of time and energy, and commercial apparatus can be costly. Moreover, the time-consuming nature of heat sterilization makes the method inconvenient in many situations, including when doctors make their rounds in hospitals.

For example, during dry heat sterilization, medical instruments are wrapped in special paper that indicates when the sterilization is complete. This type of device is typically slow, with sterilization taking on the order of an hour, although there have been some advances in technology to reduce this time to approximately 6 minutes. Steam sterilization using an autoclave is probably the most widely used type of device for sterilization. Similar to the dry heat method, instruments are first packed in special paper. The package is then placed in a high pressure/high temperature steam bath. Steam penetrates all surfaces of the instrument. At the end of the process, the steam is removed from the chamber and the device comes out dry. Autoclaving, however, is also slow and can be detrimental to some plastics. Finally, boiling and pasteurization can be used when a high degree of sterilization is not needed, however, both techniques require 10 to 20 minutes to complete.

In addition to heat-based techniques, chemical sterilization can be used in heated vapor systems as well as in cold systems. This method offers shorter times, but can be more destructive to plastics. Also, the chemical solutions used may be highly corrosive and toxic.

Germicidal infection is also a concern to parents, especially with respect to breast- and bottle-feeding equipment. Again, parents usually sterilize such equipment using steam, but a steaming apparatus is susceptible to improper use. For example, if too little water is added to the steaming apparatus, the steam does not last long enough to adequately kill the germs that may be present on the breast and bottle feeding equipment. On the other hand, if the too much water is added to the steaming apparatus, the water may not fully evaporate, and insufficient amount of steam may be generated to destroy the germs. Steam-based methods are also undesirable because they are relatively slow, which leads many parents to skip the sterilization process altogether, especially when time is of the essence.

Exposure to ultraviolet radiation is another method of killing pathogens. For years, ultraviolet light generated by mercury-vapor lamps has been used for sterilization in applications ranging from air supplied to operating rooms to the water purification in treatment plants. Its use has been limited, however, for a number of reasons, including their high cost, the tendency to produce ozone, ineffectiveness due to shadowing, and other safety concerns related to mercury and high vapor-pressure.

### SUMMARY OF THE INVENTION

Ultraviolet sterilizers consistent with this invention have many advantages over conventional technologies. First, the time needed to inactivate microorganisms is less (e.g., staph and strep can be inactivated in less than 10 seconds), no toxic chemicals are required, separate indicators (such as special paper) for degree of sterilization are not needed, and dose and time measurements can be integrated into the system.

### BRIEF DESCRIPTION OF THE DRAWINGS

The above and other advantages of the invention will be apparent upon consideration of the following detailed description, taken in conjunction with the accompanying drawings, in which:

FIG. 1 shows a perspective view of a sterilization system consistent with this invention;

FIG. 2 shows a partial cross-sectional view of the sterilization system of FIG. 1 consistent with this invention;

FIG. 3 shows a top planar view of the sterilization system of FIG. 1, with the lid open, consistent with this invention;

FIG. 4 shows a simplified block diagram of the sterilization system shown in FIG. 1 consistent with this invention; and

FIG. 5 shows another sterilization system for sterilizing baby bottles and the like consistent with this invention.

### DETAILED DESCRIPTION OF THE INVENTION

Consistent with this invention, methods and apparatus are provided for rapidly sterilizing medical tools, dental tools, and body art tools, such as stethoscopes, otoscopes, dental picks, retractors, piercing equipment, and bandage materials. Methods and apparatus are also provided for rapidly sterilizing breast- and bottle-feeding equipment using ultraviolet radiation, such as breast pumps, baby bottles, pacifiers, and the like.

Also, relatively small sterilization devices with multiple ultraviolet light-emitting sources, such as ultraviolet light-emitting diodes, multi-path mirroring and/or steering are provided. These devices can include a combination of anti-pathogen surfaces and relatively rapid surface sterilization methods to yield exceptional long-term sterilization of surface cracks, crevices, and material junctions. Internally ultraviolet reflective systems combined with active dose monitoring ensures proper deactivation of surface pathogens.

Medical/Dental/Body-Art Tool Sterilization System

Consistent with one aspect of this invention, a rapid sterilization system (e.g., for stethoscopes) is provided. The system can, for example, generate ultraviolet light using one or more mercury-vapor lamps in a sealed chamber. To prevent the generation of ozone, the ultraviolet light can be filtered to remove wavelengths below 200 nm. Intensity levels can be programmed to deliver intensities to achieve a predetermined kill of common pathogens in a matter of seconds. For example, a stethoscope consistent with one aspect of this invention can be sufficiently sterilized between about five and about ten seconds (e.g., at about 8000 uW*sec/cm²) using a lamp that is commonly marketed as a germicidal lamp. Such lamps are sold by many companies, including the General Electric Company, of Fairfield, Conn., and Royal Philips Electronics, of Amsterdam, The Netherlands.

Locking systems to ensure that the unit is properly closed during operation to prevent ultraviolet light from escaping the sealed container are also provided. For example, by applying a positive or negative pressure gas in the killing zone and monitoring the pressure to ensure an “air-tight” seal, can be used. Also, static and dynamic reflector systems can be included to distribute the ultraviolet light more uniformly to all sides and surfaces of the stethoscope. Detection systems will assure adequate ultraviolet doses.

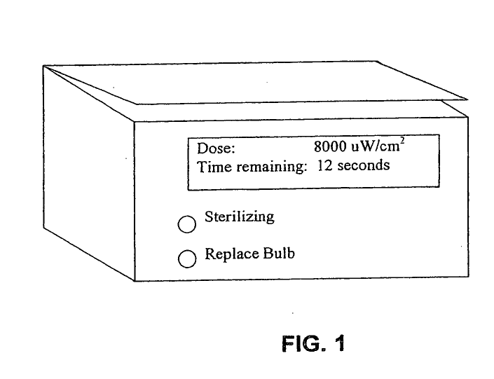

FIGS. 1-4 show various views of a sterilization system consistent with this invention. As shown in FIG. 1, the basic design includes a box-like housing in which articles are placed. Once the lid is closed, the ultraviolet light source (e.g., mercury-vapor lamp) can be turned on to generate ultraviolet light. As mentioned above, a safety mechanism, possibly based on pressure, can ensure that ultraviolet rays are contained in the box. The face of the unit can include indicators, for example that display the dose and sterilization time remaining. The front of the unit can also include a bulb replacement indicator.

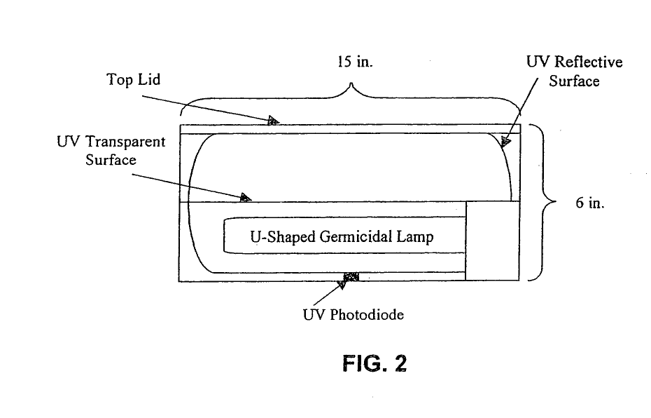

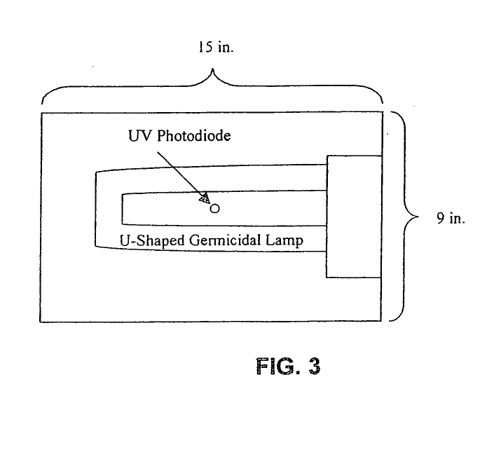

FIG. 2 shows a partial cross-sectional view of the sterilization system of FIG. 1 consistent with this invention and FIG. 3 shows a top planar view of the same system with the lid open. It will be appreciated that the shape and size of the unit can vary depending on the particular application and the light and power sources used. As shown in FIG. 3, a U-shaped germicidal lamp can be housed under an ultraviolet light transparent material, such as an ultraviolet light transparent glass, to ensure that the inner surface of the killing zone is easily cleaned. The killing zone (i.e., irradiation volume) can be about 9×15×3 cubic inches, large enough for stethoscopes and other medical instruments.

Alternatively, the unit can be sufficiently large to only insert the distal end of the stethoscope. This could dramatically reduce the size the unit housing and the time required to deliver an adequate radiation dose. In this case, the killing zone could be a small as, for example, about 2×2×2 cubic inches. In this case, the unit could provide a light-tight seal around the stethoscope cord. When mounted at neck level, a doctor could (e.g., when entering a patient's room) sterilize the stethoscope by placing the distal sensor end in the killing zone without removing the proximal, ear-plug end from around his or her neck.

A silicon carbide photodetector can be mounted in the killing zone (e.g., below the lamp) and used in a feedback circuit to determine: (a) the time needed for adequate sterilization and (b) the ultraviolet efficiency of the light source. Finally, the highly reflective surface will surround the irradiation volume to ensure multiple paths for more complete irradiation.

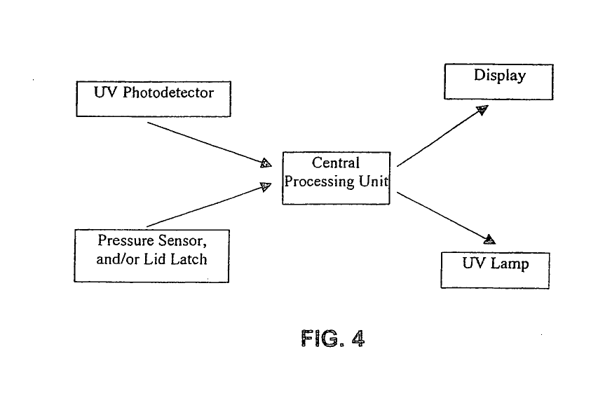

As shown in FIG. 4, the sterilization apparatus can include a central processing unit, which may be an inexpensive microprocessor. During operation, the unit can receive inputs from the photodetector and the interlock system. The unit can also control the germicidal lamp and drive the display unit.

Because significant exposure to ultraviolet light can degrade many materials, objects that are sterilized consistent with this invention, such as stethoscopes and otoscopes, gloves, gauze, and other medical items that may be used during surgery should be made from substantially ultraviolet light-resistant materials. Examples of such materials include glasses, metals, silicones, and ultraviolet light-resistant polymers.

Breast- and Bottle-feeding Equipment Sterilization System

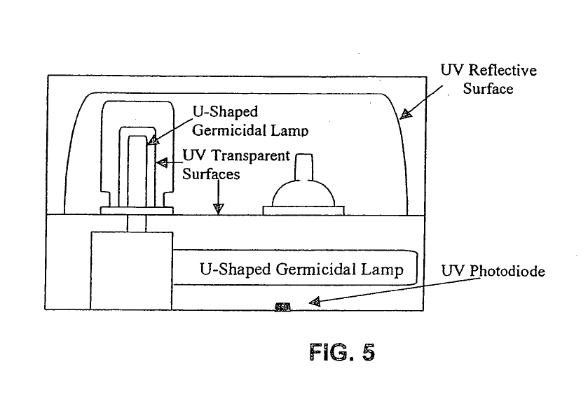

FIG. 5 shows another sterilization system for sterilizing baby bottles and the like consistent with this invention. A breast- and bottle-feeding sterilization system can use many of the technologies discussed above, including, for example, one or more high-power germicidal lamps to generate ultraviolet radiation, a highly ultraviolet-light reflective inner surface, an ultraviolet light photodetector for monitoring the dose.

Consistent with this invention, breast- and bottle-feeding equipment that is substantially transparent to ultraviolet light is provided. By making the equipment transparent, shadowing effects are substantially eliminated, and the sterilization chamber (i.e., killing zone) design can be similar to the design using for the stethoscope sterilizing apparatus shown in FIG. 1, for example. If the breast and bottle-feeding equipment is not substantially transparent, the shape of the killing chamber should be modified to ensure proper exposure to inner surfaces of the equipment. For example, a baby bottle would require ultraviolet light be shone into the bottle, which could be accomplished using an elongated light-emitting object that can be inserted into the bottle. The object can be a U-shaped germicidal lamp, a stick with one or more light-emitting diodes mounted thereon, etc.

As shown in FIG. 5, a breast and bottle-feeding sterilization system could include two or more compartments. In one compartment, an ultraviolet light source can be located above the sterilization chamber and the objects to be sterilized can be placed on a rotating plate, in a manner similar to microwave oven, to reduce the effects of shadowing. A second compartment could house the bottle-sterilizing chamber, which can include at least one vertically mounted bulb or light pipe to irradiate the inside of the bottle.

The equipment being sterilized can be formed from ultraviolet-resistant and/or transparent materials. In one embodiment, equipment surfaces can be coated with an ultraviolet resistant coating, such as is described in U.S. Pat. Nos. 6,355,189 and 6,106,605. For example, Scotchtint™ Panther Black Auto Film, which claims to block 99% of ultraviolet rays, is available from 3M, of St. Paul, Minn.

Another solution would be to use materials that are designed to be inherently resistant to ultraviolet light. This can be done by adding a stabilizing UV additive to the polymer. For example, U.S. Pat. No. 6,337,362 describes one such additive. There are also classes of glass that are resistant to ultraviolet light. Also, one or more parts of the equipment could include metal, such as aluminum. Finally, the problem can be solved by using materials that are transparent to ultraviolet light wavelengths, such as quartz.

Acrylic also adds some possibilities for both UV transparent and UV resistant materials. For example, CRYO Industries, of Rockaway, N.J., currently markets a number of different ultraviolet transparent and resistant acrylics under the name ACRYLITE®.

A bottle nipple can be fabricated out of ultraviolet light-resistant silicone. One such silicone, Silopren® LSR 2940TP 3822, is currently available from a joint venture formed between General Electric and Bayer. This product has a high resistance to both ozone and ultraviolet light.

An example embodiment of an ultraviolet light-resistant bottle could be a glass, e.g., PYREX®, bottle with a metal cap that holds an ultraviolet light-resistant plastic nipple in place. Other embodiments, including disposable nipples and caps, only require that the bottle to be sterilized.

Preferably, objects that are sterilized consistent with this invention are made with as few shadowed regions and surface cracks as possible. Moreover, antimicrobial materials, such as silvers, can be disposed on surfaces that are shadowed and difficult to reach with treat with ultraviolet light. It will be appreciated that the methods consistent with this invention can also be used in the home and office for other products, such as cutting boards or keyboard surfaces. Other applications consistent with this invention include positive re-sterilization of prepackaged materials, such as bandages or gauze. In addition, the ultraviolet light sterilization methods consistent with this invention can be used to sterilize toothbrushes and the like.

One skilled in the art will appreciate that the present invention can be practiced by other than the described embodiments, which are presented for purposes of illustration and not of limitation. The present invention is limited only by the claims that follow.

That which is claimed:

1. An ultraviolet sterilization apparatus for sterilizing objects, wherein the apparatus comprises:
a housing having an opening through which objects can be inserted for sterilization;
a killing chamber having an inner surface that is substantially reflective at ultraviolet wavelengths; and
at least one ultraviolet radiation source inside the housing.

2. The apparatus of claim 1 further comprising a photodetector mounted within the killing chamber.

3. The apparatus of claim 1 wherein the object is selected from a group consisting of a medical tool, a dental tool, a body art tool, and any combination thereof.

4. The apparatus of claim 1 further comprising an external display unit.

5. The apparatus of claim 1 wherein the killing chamber inner surface is adapted for receiving a distal end of a stethoscope.

6. The apparatus of claim 1 wherein the killing chamber inner surface is removable, and wherein the inner surface is selected from a group of differently shaped inner surface, each of said differently shaped inner surfaces being adapted to receive a different type of tool.

7. The apparatus of claim 1 wherein the object is a piece of baby feeding equipment.

8. The apparatus of claim 1 wherein the source comprises at least one light emitting diode.

9. A method of sterilizing breast- and bottle-feeding equipment comprising exposing the equipment to ultraviolet light.

10. A method of sterilizing a tool comprising exposing the tool to ultraviolet light.

11. The method of claim 10 wherein the tool is selected from a group consisting of a medical tool, a dental tool, and a body-art tool.

12. The method of claim 10 wherein the tool is a stethoscope.
